(19) United States
(12) Patent Application Publication
(10) Pub. No.: US 2011/0195988 A1
(43) Pub. Date: Aug. 11, 2011

(54) Pharmaceutical Composition

(75) Inventor: Graham Alan March, Surrey (GB)

(73) Assignee: Special Products Limited, Weybridge (GB)

(21) Appl. No.: 13/021,401

(22) Filed: Feb. 4, 2011

(30) Foreign Application Priority Data
Feb. 5, 2010 (GB) 1001911.5

(51) Int. Cl. A61K 31/485; A61P 25/04; A61P 31/10

(52) U.S. Cl. 514/282

(57) ABSTRACT

A liquid composition for administration to the buccal cavity of a patient comprising less than 10 mg/ml morphine and a pharmaceutically acceptable carrier

This invention relates to a pharmaceutical composition. In particular it relates to a liquid pharmaceutical composition for the buccal administration of morphine and the use of such compositions in preventing the onset or treatment of breakthrough pain as well as the treatment of other types of moderate to severe acute pain.

Morphine is an opioid analgesic which has been used widely since its discovery in 1817. Morphine is considered to be one of the most valuable analgesic drugs for the treatment of severe pain, despite its tendency to cause nausea and vomiting. It is a versatile drug which is administered to patients via a range of routes, depending on the needs of the patient.

Morphine may be directly administered intramuscularly, intravenously or subcutaneously to a patient using a syringe. This route of administration is preferable where a rapid-onset, high dose of morphine is required. However, the administration to patients, especially children, of drugs via a needle may increase anxiety levels. Additionally, for patients receiving repeated doses of a number of different drugs via the intravenous or intramuscular route, the location of a viable injection site may prove challenging. Further, injectable morphine is short acting and thus, its use is not appropriate for patients suffering from prolonged episodes of pain.

A prolonged analgesic effect of constant intensity may be achieved by intravenously drip-feeding morphine to patients. This route of administration is preferable where a patient is in a relatively constant level of pain. The rate of delivery of the drug to the patient can be controlled to ensure that the patient's level of analgesia is appropriate, i.e. the patient is in a state of relative comfort without any loss of consciousness. While the patient will be comfortable when the level of pain is constant, there will be instances where the patient's pain level is temporarily increased. For example, such instances may arise when a patient's dressing or cannula, if used, is changed, when the patient gets out of bed, or when there is a temporary surge in the patient's pain level for any other reason. In these situations, the level of analgesia provided by the intravenous drug will not be sufficient to maintain the patient's comfort level. These types of episodes of pain are known as breakthrough pain. Episodes of breakthrough pain are suffered by a broad range of patient groups, and are commonly suffered by cancer patients.

Attempts to treat breakthrough pain using morphine have been made, for example using a central line which is provided with means for the patient to control a supply of morphine which can be administered when an additional dose of analgesic is required. The use of such apparatus, however, limits the patient's motility and also requires the presence of a skilled medical professional to set the apparatus up.

A prolonged analgesic effect can also be attained through the oral administration of morphine, which has been formulated in extended release tablets, capsules, oral solutions and oral granules. Due to its relatively short half life, morphine must be administered every four hours if the immediate release oral formulations are used. The presence of a variety of modified and extended release oral formulations has limited the role of immediate release preparations for the relief of acute pain.

The oral administration of morphine does not require the presence of a healthcare professional, and thus, oral formulations of morphine can be administered at home by the patient. However, there is a time lag in the onset of analgesia as the drug is absorbed; the analgesic effect is only observed after around 30 minutes following administration. Thus, these formulations are not ideal for the treatment of breakthrough pain, especially in paediatric patients. Further, morphine undergoes extensive first pass metabolism, resulting in only 30-40% of an orally administered dose being bioavailable. Additionally, the administration of solid dosage forms, for example, tablets or capsules, presents a choking risk to patients and are therefore not suitable dosage forms for children or elderly patients who have swallowing difficulties.

Limited research into the administration of morphine via the buccal route has been conducted. Of the research that has been conducted, there is a general consensus that the buccal administration of morphine is challenging and ineffective, for example Simpson et al., Br. J. Clin. Pharmacol. 1989 March; 27(3): 377-380; Hoskin et al., Br J Clin Pharmacol. 1989 April; 27(4): 499-505; Fisher et al., Br J Clin Pharmacol. 1987 December; 24(6): 685-687.

The administration of injectable formulations of morphine into the buccal cavity of patients is known. The use of injectable morphine in this way is problematic for several reasons. Firstly, the dosage of the injectable formulation is calculated on the assumption that the drug will be administered intravenously. A different quantity of morphine will enter the bloodstream when the injectable formulation is administered buccally, due to a loss of the drug when crossing the buccal membrane, as opposed to when it is injected.

Additionally, only a certain proportion of the injectable formulation will be available for absorption through the buccal membrane. A quantity will trickle towards the back of the throat and be swallowed, resulting in a two phase onset of analgesia.

Further, morphine which is formulated for intravenous injection is a controlled substance. As a requirement of legislation in most countries, supplies of such drugs are carefully controlled. For example, in the UK, a formulation containing morphine at a concentration over 2 mg/ml is considered to be a controlled drug and must be stored in a locked cabinet. If a patient is in urgent need of analgesia, locating and accessing the supply of the drug will add to the time required to prepare and administer the dose.

Once the healthcare professional has accessed the injectable morphine, there will be a further delay as the buccal dose is prepared. This involves drawing the liquid out of an ampoule using a syringe with a needle and transferring that liquid to an oral syringe before it can be administered to the buccal cavity of a patient.

Finally, the taste of morphine is unpleasant meaning that the acceptance of patients, especially children, to injectable morphine administered via the buccal route is low.

Accordingly, there remains a need in the art for a formulation of morphine which achieves one or more of the following aims: the formulation may be used to prevent the onset of episodes of breakthrough pain, the formulation may be used to treat episodes of breakthrough pain, the formulation may be used to treat episodes of moderate to severe acute pain, the formulation has rapid onset, administration of the formulation is not distressing to patients, the formulation is acceptable to all patient groups, the formulation has a high rate of drug absorption, administration of the formulation is straightforward, a low level of training to healthcare professionals or carers is required to enable them to administer the formulation, the formulation can be administered outside of a hospital, the formulation presents no choking risk, the formulation is suitable for administration to children including infants.

The failure to provide medicaments achieving these aims and other problems experienced in the art are solved by the present invention.

### SUMMARY OF THE INVENTION

According to a first aspect of the present invention there is provided a liquid composition for administration to the buccal cavity of a patient comprising morphine at a concentration of less than 10 mg/ml and a pharmaceutically acceptable carrier.

Breakthrough pain is usually foreseeable. The formulation of the present invention provides a rapid onset of analgesia and thus can be administered shortly before the action which is expected to cause breakthrough pain, e.g. the patient leaving his bed, or the patient's dressing being changed or removed. In this way, the onset of breakthrough pain can be reduced, or ideally, prevented.

However, the spontaneous onset of breakthrough pain may also occur. Spontaneous episodes of breakthrough pain are more common in certain patient groups, including cancer patients. As a result of the rapid onset of the formulations of the present invention, they may also be used to treat episodes of breakthrough pain, where those episodes occur unexpectedly.

The time between administration of the formulation of the present invention and the onset of analgesia is less than 30 minutes, i.e. the onset of analgesia is more rapid when the formulations of the present invention are used than when oral solutions of morphine are administered. Preferably, the onset of analgesia takes less than ten minutes, and more preferably less than five minutes.

Additionally, the buccal route is a non-invasive and relatively straightforward route of administration. A limited amount of training is required for healthcare professionals or carers to become competent to administer medicaments in this way.

Presently commercialised injectable formulations of morphine have concentrations that range from 10 mg/ml to 30 mg/ml. It has surprisingly been found that doses of lower concentrations of morphine can be used to reduce, and ideally prevent the onset of breakthrough pain when the formulation of the present invention is administered via the buccal route. The formulations of the present invention can also be used to treat spontaneously occurring episodes of breakthrough pain as well as other types of episodes of moderate to severe acute pain. Accordingly, the concentration of morphine in the formulation of the present invention is less than about 10 mg/ml. In preferred embodiments of the present invention, the concentration is from about 0.1 to about 5 mg/ml, about 0.5 to about 4 mg/ml or from 1.0 to 3.0 mg/ml. In a most preferred embodiment, the concentration of the formulation is about 2.0 mg/ml.

The use of lower concentrations of morphine is advantageous, not only because the overall load of opioid administered to patients (especially children) is reduced, but also because the regulations governing the handling of formulations with lower concentrations of morphine are more relaxed. For example, in the UK, formulations of morphine with a strength equal to or less than 0.2% (i.e. 2 mg/ml) are ‘Schedule 5’ controlled drugs, meaning that the requirement to store them in a controlled medicines cabinet, which applies to higher strength products, does not apply. Accordingly, formulations of the present invention will be more accessible to healthcare professionals, which will improve the ease of obtaining and administering those formulations to patients in need thereof.

The compositions of the present invention preferably have a viscosity of about 500 CP or lower when measured at 22° C. In preferred embodiments, the viscosity may be about 200 to 400 CP, about 250 CP to 350 CP, or most preferably, about 270 CP to 330 CP.

Viscosity may be measured using any apparatus known to those skilled in the art, for example, Brookfield LVDV +/−.

The use of a formulation having a viscosity of from about 200 CP to about 400 CP is advantageous as when it is administered into the buccal cavity of a patient, there is a low degree of circulation of the formulation throughout the mouth, meaning that the risk of a quantity of the formulation trickling down the patient's throat is minimised, if not eliminated.

The viscosity of the formulation may inherently be provided by the other excipients included therein, or a viscosity enhancing component may additionally be present. Preferred viscosity enhancing components include glycerol, carrageen, quince seed, casein, dextrin, gelatin, carboxy vinyl polymer, hydrogenated starch hydrolysate, maltitol syrup, sugar (dextrose, glucose and sucrose), cellulose derivatives such as sodium or calcium carboxymethylcellulose, hydroxy ethyl cellulose and hydroxypropyl cellulose, a polysaccharide, a pectin, agar, a hydrophilic gum such as acacia gum, guar gum, arabic gum and xanthan gum, tragacanth gum, alginic acid, an alginate, dextran, a carbomer resin or mixtures thereof. Glycerol is especially preferred as, in addition to enhancing viscosity, it has been advantageously found to stabilise morphine.

The pH of the formulation is preferably about 2 to about 11. In a more preferred embodiment, the pH ranges from 8 to 10, most preferably pH 9. Approximately neutral and slightly basic pHs are generally preferred as the absorption of morphine at these pHs has been found to be optimal. This is believed to be because the non-ionised form of morphine, which is lipophilic, is prevalent at these pHs and is more easily absorbed through the buccal membrane.

However, as the rate of degradation of morphine increases with pH, an acidic pH of, for example, 2 to 6, or more preferably, from 4 to 6 may be preferred in some arrangements to improve the stability of morphine. As a result of the advantageous nature of the formulation of the present invention, an efficacious dose of morphine will still be administered when a formulation having a lower pH is employed.

Excessively high pHs, for example, above 11 are generally avoided as the rate of degradation of morphine is increased and also because formulations at such high pHs will have an unacceptable ‘soapy’ taste.

The pH of the formulation may be inherently provided by the excipients present in the formulation or a pH adjustment agent may be employed. The pH adjustment agent may comprise a simple base or acid, or may additionally or alternatively comprise a buffer.

Where the formulation is formulated as an alkali, the use of a buffer is preferred as, once the formulation is administered to the buccal cavity, the buffer will maintain the desired pH of the formulation whereas the pH of a simple basified formulation will rapidly be adjusted to around 7 by saliva once administered. Any pharmaceutically acceptable buffer may be employed, although buffers which maintain the pH of the formulation at a roughly neutral or slightly basic pH are preferred. Examples of such buffers include phosphate buffers, glycine/NaOH buffers, carbonate or bicarbonate buffers.

However, in arrangements where the formulation is acidic, simple acidification, rather than the use of an acidic buffer, is preferred. This is because, once administered, the pH of the formulation will be rapidly increased to around 7 by saliva. As mentioned above, the absorption of morphine is greater at neutral pH than at acidic pHs and thus, the pH increase imparted by saliva will result in an increase in the absorption of morphine by the patient.

To increase shelf-life, the formulation may include a microbial preservative. Any preservative which does not adversely interact with morphine or any of the excipients may be employed. Preferred preservatives include alcohol, benzalkonium chloride, benzethonium chloride, benzoic acid, benzyl alcohol, bronopol, butyl-paraben, cetrimide, chlorhexidine, chlorobutanol, chlorocresol, cresol, ethanol, ethylparaben, glycerin, imidurea, methylparaben, phenol, phenoxyethanol, phenylethyl alcohol, phenyl mercuric acetate, phenyl mercuric borate, phenylmercuric nitrate, potassium sorbate, propylene glycol, propyl-paraben, sodium benzoate, sodium propionate, sorbic acid and thiomersal or a mixture thereof. The amount of preservative may range, for example, from about 0.5 to about 10 mg/ml of the composition, preferably from about 1 to about 5 mg/ml of the formulation.

Shelf life may also be increased by preventing the oxidation of morphine. This may be achieved by limiting exposure of the formulation to light. For example, if the formulation of the present invention is provided as a bulk solution, it would be preferable for that solution to be packaged in a dark-coloured glass bottle. Alternatively, if the formulation is to be provided in unit dosage form, then the dosage forms are preferably overwrapped with an opaque packaging material.

Additionally, antioxidants, such as sodium metabisulphite, ascorbic acid, or chelating agents, such as sodium edetate, may be employed. Glycerine may also be used to act as a stabilising agent. The amount of antioxidant and stabilising agent may range, for example, from about 0.5 to about 10 mg/ml of the composition, preferably from about 1 to about 5 mg/ml of the formulation.

The formulation may also contain an antifungal agent. Typical antifungal agents include alkali metal salts of an alkyl hydroxybenzoate, such as sodium methyl hydroxybenzoate, sodium propyl hydroxybenzoate, or mixtures thereof. The amount of antifungal agent may range for example, from about 0.5 to about 1 mg/ml of the composition, preferably from about 1 to about 5 mg/ml of the formulation.

Morphine has an unpleasant taste. To improve the acceptance of the formulation of the present invention, a sweetening agent is preferably employed. Preferred sweetening agents include sugar, acesulfame, sucralose, high fructose corn syrup, maltitol syrup, cyclamates, saccharins and aspartame. However, as the formulation is intended to be held in the buccal cavity, adjacent to the teeth, the sweetening agent is preferably a synthetic sweetening agent.

A flavour enhancer may also be employed in the formulations of the present invention. The flavour enhancer may impart any flavour to the formulations which improves their acceptance by patients. Preferred flavours include strawberry, raspberry, cranberry, banana, peach, mango, passion fruit, apple, grape, caramel, watermelon, chocolate, coffee, yoghurt, vanilla, ice cream or bubblegum.

The amount of sweetening agent and/or flavour enhancer preferably ranges from about 10 mg/ml to about 100 mg/ml of the formulation.

Any pharmaceutically acceptable carrier may be included in the formulation of the present application. The carrier may comprise water, alcohols (including monohydric alcohols e.g., ethanol and polyhydric alcohols, e.g., glycerine, glycerol and non-toxic glycols such as polyethylene glycol or propylene glycol) and their derivatives, and oils (e.g., fractionated coconut oil and arachis oil) or mixtures thereof. Ethanol is an especially preferred carrier as it has advantageously been found to retain morphine base in solution for a long enough time to ensure a high rate of absorption into the buccal membrane and also act as a microbial preservative.

Any pharmaceutically acceptable and effective form of morphine may be included in the formulation of the present invention. For example, the free base of morphine may be employed. Alternatively, a salt may be used. In preferred embodiments of the present invention, the hydrochloride, sulphate, tartrate, acetate, or citrate salt is used. The sulphate salt is the most commonly used form of morphine. However, the chloride salt is also advantageous for morphine administered via the buccal route as it is more lipophilic and is therefore be more readily absorbed across the buccal membrane. Pharmaceutically acceptable salts include, but are not limited to, hydrochloride, hydrobromide, hydroiodide, nitrate, sulphate, besylate, bisulphate, phosphate, acid phosphate, propionate, isonicotinate, acetate, lactate, salicylate, citrate, tartrate, pantothenate, bicarbonate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

The formulations of the present invention may be administered sequentially or simultaneously with other therapeutic agents selected from (but not limited to) corticosteroids, cytotoxics, antibiotics, immunosupressants, nonsteroidal antiinflammatory drug, other narcotic analgesics, local anaesthetics, NMDA antagonists, neuroleptics, anticonvulsants, antispasmodics, antiemetics, antidepressants or muscle relaxants. The agents can be administered separately or in combination. The formulations of the present invention may be provided as a bulk solution, from which doses can be removed. However, in a preferred embodiment, the formulation is provided as a unit dose. The unit dose is preferably provided in a single-use means of administration, most preferably a dropper, such as that disclosed in UK Patent Application No. 0922357.9, the contents of which are incorporated by reference.

The unit dose may comprise between about 0.1 to about 20 ml of medicament. In more preferred embodiments, the unit dose comprises about 0.2 to about 10 ml, about 0.25 to about 5 ml, or more preferably, about 0.5 to about 2 ml of the formulation. In a most preferred arrangement, the unit dose comprises 1 ml of medicament. The use of a unit dose of medicament is advantageous as the risk of administering an incorrect dose is eliminated.

The formulation of the present invention may be provided as part of a kit. The kit may also comprise instructions to administer the formulation to the buccal cavity of a patient in need thereof. The formulation is preferably included in the kit in the form of at least one unit dose of the formulation of the present invention. The kit may also comprise one or more doses of a further therapeutic agent.

According to a second aspect of the present invention, there is provided the use of morphine in the manufacture of a liquid medicament for administration via the buccal cavity of a patient to prevent the onset of breakthrough pain or to treat episodes of moderate to severe acute pain, including breakthrough pain.

Besides breakthrough pain, other types of episodes of moderate to severe acute pain include (but are not limited to) pain associated with cancer, surgery, arthritis, dental surgery, trauma, musculoskeletal injury or disease, visceral diseases, painful bladder syndrome) and migraine headache. Additionally the painful conditions can be neuropathic (post-herpetic neuralgia, diabetic neuropathy, drug-induced neuropathy, HIV-mediated neuropathy, sympathetic reflex dystrophy or causalgia, fibromyalgia, myofacial pain, entrapment neuropathy, phantom limb pain, trigeminal neuralgia). Neuropathic conditions include central pain related to stroke, multiple sclerosis, spinal cord injury, arachnoiditis, neoplasms, syringomyelia, Parkinson's and epilepsia.

According to a third aspect of the present invention, there is provided the use of morphine to prevent the onset of breakthrough pain or to treat episodes of moderate to severe acute pain, including breakthrough pain, wherein the morphine is administered as a liquid medicament to a patient via the buccal cavity.

According to a fourth aspect of the present invention, there is provided a method of preventing the onset of breakthrough pain or treating episodes of moderate to severe acute pain, including breakthrough pain by administering the formulations discussed above to the buccal cavity of a patient.

In these second, third and fourth aspects of the present invention, the morphine is preferably administered in the formulation as described above.

It is clearly advantageous for the formulations of the present invention to be administered prior to the onset of breakthrough pain, in order to prevent the onset of breakthrough pain. However, the formulations of the present invention can also be used to treat breakthrough pain in the event that the episode of breakthrough pain was not foreseeable (i.e. it was spontaneous) or if the formulation was not administered in time, prior to the onset of breakthrough pain. The formulations of the present invention can also be used to treat other types of episodes of moderate to severe acute pain, besides breakthrough pain.

The formulations of the present invention are suitable for use with all patient groups. However, certain advantages are especially apparent when the formulations are used with paediatric patients. For example, administration of morphine via the buccal route allows the onset of breakthrough pain to be prevented while avoiding causing anxiety to patients through the use of a syringe, which will be especially acute for such younger patients. Additionally, the use of formulations having acceptable flavour profiles will be especially important for such patients.

The term ‘paediatric patient’ covers all children and adolescents below the age of 18. The to formulations of the present invention are especially suitable for younger patients, e.g. pre-term infants to children of 12 to 14 years of age.

Where the concentration of morphine is provided, e.g. the formulation has a concentration of less than 10 mg/ml, it is the concentration of free morphine base which is provided and not the concentration of any salt of morphine that may be employed.

Where ‘mg/ml’ units are used to define the concentrations of morphine and excipients, the concentrations are provided as a proportion of the total formulation.

The invention is further illustrated in the following Examples.

### EXAMPLE 1

### Formulation Having Improved Absorption

As mentioned above, the absorption of morphine into the buccal membrane is greater at neutral or mildly basic pHs. Accordingly, the following formulation was prepared with the aim of maximising the absorption of morphine:

| Component | Concentration |
|---|---|
| Morphine sulphate | 0.2% |
| | (as anhydrous base) |
| Ethanol | 15% |
| Glycerol | q.s. to adjust viscosity |
| Aspartame | 0.2% |
| Sodium Hydroxide | To pH9 |
| Sodium Edetate | 0.1% |
| Water | q.s. to adjust viscosity |

### EXAMPLE 2

### Buffered Formulation Having Improved Absorption

| | |
|---|---|
| Morphine sulphate | 0.2% |
| | (as anhydrous base) |
| Ethanol | 15% |
| Glycerol | q.s. to adjust viscosity |
| Aspartame | 0.2% |
| Sorensen | To pH9 |
| (glycine/NaOH) | |
| buffer | |
| Sodium Edetate | 0.1% |
| Water | q.s. to adjust viscosity |

### EXAMPLE 3

### Formulation Having Improved Stability

The rate of degradation of morphine increases with pH. While a neutral or slightly basic pH (e.g. 7 to 11) is preferable as the absorption of morphine is maximised within this range, it may be necessary, in certain embodiments, to formulate at a lower pH to improve stability and shelf life of the formulation. The following formulation is an example of an acidic formulation of the present invention:

| Component | Concentration |
|---|---|
| Morphine sulphate | 0.2% |
| | (as anhydrous base) |
| Ethanol | 15% |
| Glycerol | q.s. to adjust viscosity |
| Aspartame | 0.2% |
| Sulphuric Acid | to pH4 |
| Sodium Edetate | 0.1% |
| Water | q.s. to adjust viscosity |

1. A liquid composition for administration to the buccal cavity of a patient comprising less than 10 mg/ml morphine and a pharmaceutically acceptable carrier

2. The composition of claim 1, wherein the composition has a viscosity of about 200 CP to about 400 CP measured at 22° C.

3. The composition of claim 1 or 2, wherein the concentration of morphine is about 1 to about 5 mg/ml.

4. The composition of any one of claims 1 to 3, wherein the concentration of morphine is about 0.5 to about 4 mg/ml.

5. The composition of any one of claims 1 to 4, wherein the viscosity of the composition is about 250 CP to about 350 CP

6. The composition of any one of claims 1 to 5, wherein the viscosity of the composition is about 270 CP to about 330 CP.

7. The composition of any one of claims 1 to 6, wherein the composition further comprises a viscosity enhancing component.

8. The composition of any one of claims 1 to 7, wherein the pH of the composition is about 2 to about 11.

9. The composition of any one of claims 1 to 8, wherein the pH of the composition is about 7 to about 11.

10. The composition of any one of claims 1 to 9, wherein the composition further comprises a buffer.

11. The composition of claim 10, wherein the buffer is a phosphate buffer, a glycine/NaOH buffer or a carbonate or bicarbonate buffer or a mixture thereof.

12. The composition of any one of claims 1 to 11, wherein the composition additionally comprises a sweetener, a flavour enhancer, a preservative and/or an antifungal agent.

13. The composition of any one of claims 1 to 12, wherein the carrier comprises water, ethanol, glycerine, glycerol, polyethylene glycol, propylene glycol or mixtures thereof.

14. The composition of any one of claims 1 to 13, further comprising an additional therapeutic agent.

15. The composition as hereinbefore described in the accompanying description.

16. A unit dose of the composition of any one of claims 1 to 15.

17. The unit dose of claim 16, comprising about 0.1 to about 10 ml of the formulation.

18. The unit dose of claim 16 or 17, comprising about 0.2 to about 5 ml of the formulation.

19. The unit dose of any one of claims 16 to 18, comprising about 0.5 to about 2 ml of the formulation.

20. The unit dose of any one of claims 16 to 19, wherein the unit dose is contained within a single-use means of administration.

21. The unit dose of any one of claims 16 to 20, wherein the single-use means of administration is a dropper.

22. A kit comprising the composition of any one of claims 1 to 15 or the unit dose of any one of claims 16 to 21 and instructions to administer the formulation into the buccal cavity of a patient.

23. The kit of claim 22, further comprising an additional therapeutic agent.

24. The kit of claim 23, wherein the additional therapeutic agent is provided as a unit dose.

25. The use of morphine in the manufacture of a liquid medicament for administration via the buccal cavity of a patient to prevent the onset of breakthrough pain wherein the medicament comprises less than 10 mg/ml morphine.

26. The use of morphine in the manufacture of a liquid medicament for administration via the buccal cavity of a patient to treat episodes of moderate to severe acute pain, including breakthrough pain wherein the medicament comprises less than 10 mg/ml morphine.

27. The use of morphine to prevent the onset of breakthrough pain, wherein the morphine is administered in a liquid medicament to a patient via the buccal cavity and the medicament comprises less than 10 mg/ml morphine.

28. The use of morphine to treat episodes of moderate to severe acute pain, including breakthrough pain, wherein the morphine is administered in a liquid medicament to a patient via the buccal cavity and the medicament comprises less than 10 mg/ml morphine.

29. The use of any one of claims 25 to 28, wherein the medicament has a morphine content of 2 mg/ml or less

30. The use of any one of claims 25 to 29, wherein the medicament comprises the formulation of any one of claims 1 to 15.

31. The use of any one of claims 25 to 30, wherein the patient is 14 years of age or under.

32. The use of any one of claims 25 to 31, wherein the patient is simultaneously or sequentially administered an additional therapeutic agent.

33. The use of claim 32, wherein the medicament comprises the additional therapeutic agent.

34. The use of claim 32 or 33, wherein the additional therapeutic agent is selected from the group consisting of corticosteroids, cytotoxics, antibiotics, immunosupressants, nonsteroidal antiinflammatory drug, narcotic analgesics, local anaesthetics, NMDA antagonists, neuroleptics, anticonvulsants, antispasmodics, antiemetics, antidepressants and muscle relaxants.

35. A method of preventing the onset of breakthrough pain by administering morphine formulated in a liquid medicament to the buccal cavity of a patient, wherein the medicament comprises less than 10 mg/ml morphine.

36. A method of treating episodes of moderate to severe acute pain, including breakthrough pain, by administering morphine formulated in a liquid medicament to the buccal cavity of a patient, wherein the medicament comprises less than 10 mg/ml morphine.

37. The method of claim 35 or 36, wherein the medicament has a morphine content of 2 mg/ml or less.

37. The method of any one of claim 35 or 36, wherein the medicament comprises the formulation of any one of claims 1 to 15.

38. The method of any one of claims 35 to 37, wherein the patient is 14 years of age or younger

39. The method of any one of claims 35 to 38, wherein the patient is simultaneously or sequentially administered an additional therapeutic agent.

40. The method of claim 39, wherein the medicament comprises the additional therapeutic agent.

41. The method of claim 39 or 40, wherein the additional therapeutic agent is selected from the group consisting of corticosteroids, cytotoxics, antibiotics, immunosupressants, nonsteroidal antiinflammatory drug, narcotic analgesics, local anaesthetics, NMDA antagonists, neuroleptics, anticonvulsants, antispasmodics, antiemetics, antidepressants and muscle relaxants.
